(19) United States
(12) Patent Application Publication
(10) Pub. No.: US 2010/0003309 A1
(43) Pub. Date: Jan. 7, 2010

(54) METHOD FOR THE PRODUCTION OF FENTANYL-BASED ANESTHETICS FOR THEIR ORAL TRANSMUCOSAL ADMINISTRATION IN A CANDY-LIKE DOSAGE FORM

(76) Inventor: Karen Elizabeth Quiroga, Lisboa (PT)

Correspondence Address: SUGHRUE MION, PLLC, 2100 PENNSYLVANIA AVENUE, N.W., SUITE 800, WASHINGTON, DC, 20037, US

(21) Appl. No.: 12/373,195

(22) PCT Filed: Jul. 11, 2007
(86) PCT No.: PCT/PT07/00030

§ 371 Date: Apr. 22, 2009

(30) Foreign Application Priority Data
Jul. 12, 2006 (AR) P06 01 02985

(51) Int. Cl. A61K 9/00; A61K 31/445; A61P 29/00

(52) U.S. Cl. 424/440; 514/329

(57) ABSTRACT

A method for the production fentanyl-based analgesics for their oral transmucosal administration in a candy-like dosage form, involving the dissolution of a pharmacologically accepted amount of Fentanyl citrate in purified water and the dissolution of a pharmacologically accepted dye in purified water. Mix polyalcohol sugar with purified water with a ratio polyalcohol/sugar between 17 and 19.5% by weight. Stir slowly over low heat until a liquid and smooth mass is obtained. Add sucrose to this liquid mass with a ratio liquid mass/sucrose between 65% and 67.5% by weight. Heat the mass and bring to a boil from an initial temperature of 128°-1320 C to a maximum temperature of 148°-152° C., until its water content evaporates and the weight of the mass obtained in this step reaches between 88% and 90% by weight with respect to the initial total weight of the liquid mass. Leave the mass to cool to a temperature of about 1280 C, and add the Fentanyl citrate solutions, the dye and flavouring agent, stirring firmly and maintaining the same temperature. Pour this hot mixture with a temperature of about 1300 C into a mould or matrix. Before the candy mass sets, a handle can be attached to each candy piece. When the mould is cold and the candy has solidified, remove from the mould and immediately wrap or bag the pieces.

### FIELD OF THIS INVENTION

This invention concerns a new method for the production of Fentanyl-based analgesics for their oral transmucosal administration in a candy-like dosage form.

### STATE OF THE PREVIOUS ART

The use of Fentanyl derivatives to control pain as an alternative to opioids is a widely known approach, being Fentanyl citrate the most commonly used formulation for this purpose.

In connection with the above, this descriptive memory refers to the AVERY Handbook, Lippincott-Raven Publishers, 1997 Edition, that enumerates the different classes of opioids and opioid receptors and actions.

It is also known that the transmucosal administration of Fentanyl-based analgesics, in particular Fentanyl citrate, is a useful tool in the management of pain, specially the management of breakthrough pain in cancer patients. If morphine, for instance, even the immediate release type, were used to relieve pain, the analgesic effect would begin about 30 minutes after administration.

“Breakthrough pain” refers to a transitory exacerbation of pain that occurs on a background of otherwise controlled chronic pain. One of the characteristics of breakthrough pain—at least in 60 percent of cases—is its rapid onset of action, with a mean duration of effect of 33.8 minutes and high pain intensity (mean VAS 7.3). Consequently, the administration of oral morphine providing rapid absorption has not proved sufficient to relieve the pain episode, as its onset of action matches the average end of the pain peak level.

The appearance of Fentanyl preparations for oral administration has offered a solution to the problem. Its pharmaco kinetics is determined by the bioavailability and transmucosal absorption of Fentanyl, with a 25 percent absorption by this route in less than 5 minutes, and an immediate analgesic response, while the remaining 75 percent is dissolved in saliva and digested, with a slower digestive absorption. This memory also refers to the paper written by José Luis López Gonzalez et al., “Transmucosal administration of opioides”; the abstract of an article written by Russell K. Portenoy et al. in PAIN, (Pain 79 (1999) 303-312) “Oral Transmucosal Fentanyl Citrate (OTFC) for the Treatment of Breakthrough Pain in Cancer Patients”, and the article published in ONCOLOGY, August 1999, by Mary A. Simmonds, M.D., “Management of Breakthrough Pain Due to Cancer”, all of which extensively deal with and describe the above mentioned subject, and are used for reference regarding a deeper understanding of the uses and advantages of Fentanyl.

It has been widely accepted for several years that introducing a Fentanyl compound, more specifically Fentanyl citrate, into a candy matrix constitutes the most suitable and practical route for the transmucosal administration of Fentanyl derivatives. To that effect, the following material is incorporated to the descriptive memory of this invention: pages 1 and 2 of “PHARMACY HEALTH” review, the publication titled “Fentanyl Oralet” and the material published by F. A. Davis Company, the description of the Fentanyl-containing lollipop marketed under the trademark ACTIQ™, and most especially U.S. Pat. No. 4,671,953, assigned to the University of Utah Research Foundation. In fact, Janssen Pharmaceutical published an article in 1952, “Medicated Candie” concerning the pharmaco kinetics of transmucosal Fentanyl administered in a candy-like dosage form.

The examples above help to demonstrate that obtaining a soluble matrix material in a candy-like dosage form (“lollipop”) into which the drug Fentanyl has been dispersed, is widely known and already in the public domain.

However, in the course of her research, this invention assignee has come to the conclusion that harmonizing the steps involved in the obtention of said candy-like dosage form with the adequate dispersion of Fentanyl and the correct compliance with very strict parameters, constitutes a highly critical target. That is to say, optimal results cannot be achieved simply by dispersing Fentanyl within the soluble matrix material, as U.S. Pat. No. 4,671,953 intends to indicate. The examples given in said patent are very primitive and do not allow the obtention of a high-quality product.

On the contrary, based on her research, the patent assignee can offer a well founded observation that makes an important point; the simple alteration of or ambiguity in the manufacturing steps involved in the production of the analgesic-containing lollipop results in a product that fails to meet the required standards.

### OBJECTS OF THE PRESENT INVENTION

The main purpose of the present invention is to secure a method for obtaining a soluble matrix material in candy-like dosage form into which the drug Fentanyl has been dispersed, providing a uniform drug dispersion and concentration in all matrix points.

Another purpose of the present invention is to secure a method for obtaining a candy matrix containing an analgesic-effect-inducing Fentanyl compound with extended shelf-life storage without losing activity or altering its properties.

It is also a purpose of the present invention that Fentanyl citrate be the Fentanyl compound of choice.

### BRIEF DESCRIPTION OF THE INVENTION

A method for the production of Fentanyl-based analgesics and their oral transmucosal administration in a candy-like dosage form, comprising the following steps:
a) Prepare a first solution by adding purified water to a vial containing Fentanyl citrate in an amount that allows the obtention of 370 μgrams to 430 μgrams solution per candy, shaking the vial until it is completely dissolved. Set aside;
b) Prepare a second solution by adding purified water to a vial containing a pharmacologically accepted dye, shaking the vial until it is completely dissolved. Set aside;
c) Mix polyalcohol sugar with purified water with a ratio polyalcohol/sugar between 17% and 19.5% by weight. Stir slowly over low heat until a liquid and smooth mass is obtained. Add sucrose to this liquid mass with a ratio liquid mass/sucrose between 65% and 67.5% by weight, and stir until the mixture is smooth.
d) Increase heat to high and bring to boil;
e) Maintain heat from an initial temperature of 128°-132° C. to a maximum temperature of 148°-152° C., with a gradual and controlled temperature increase gradient, stirring constantly to avoid the crystallization of sugar on the rim of the reactor or container and until water content, including the water contained in the polyalcohol sugar, evaporates and until the weight of the mass obtained in this step reaches between 88% and 90% by weight with respect to the initial total weight of the liquid mass obtained in step (c);
f) Leave the mass obtained in step (e) to cool until reaching a temperature of about 5° C., stirring gently;
g) Add the solutions obtained in steps (a) and (b) and a flavouring agent, stirring firmly and maintaining the temperature reached in step (f);
h) Pour this hot mixture with a temperature of about 6° C. into a mould or matrix;
i) Before the candy mass sets, a handle can be attached to each candy piece. When the mould is cold and the candy has solidified, remove from the mould and immediately wrap or bag the pieces.

### Preferred Method for Obtaining the Invention

Here follows an illustrative example of a sample preparation of an analgesic candy of 400 μgrams and the method used to obtain the invention:

The first step is to determine the weight of a melting pot and a spatula. Next a candy mould or matrix is prepared. This generic mould or matrix is placed on a refractory plate set at 130° C.

A solution is prepared in a vial by dissolving 140 mgrams of Fentanyl citrate powder in 4 ml of purified water, shaking until the drug is completely dissolved. The complete and homogeneous dissolution of the drug is essential to obtain a uniform distribution in the candy matrix.

Next, a second solution is prepared by adding 3 ml of purified water to a vial containing a dye. For this example, the dye of choice was Amaranth Red. The vial is shaken until the dye is completely dissolved and the solution is set aside.

In a melting pot, 490 grams of polyalcohol sugar, in this case, glucose, are mixed with 90 ml of purified water, stirring slowly over low heat until a liquid and smooth mass is obtained. Next, 875 grams of sucrose are added to the mixture, stirring until it is smooth.

Next, the mass is put to boil in order to obtain the complete homogenization of the polyalcohol solution, stirring constantly to avoid the crystallization of sugar on the rim of the container (melting pot) and until water content, including the glucose water content, completely evaporates. This is a critical step of the method, water content must be evaporated until the weight of 1296.8 grams is reached, starting with an initial formula weight of 1455 gr=(190+90+875) gr. The mass must be heated gradually until it reaches a temperature of about 130°-150° C., without exceeding these parameters.

The mass is left to cool until reaching a temperature of about 130° C. stirring gently until it is completely smooth. When the mass reaches 130° C., the Fentanyl solution, the flavouring agent and the dye are added. This is also a critical step of the method to obtain this final product.

The candy mass containing the dissolved drug is poured into the above mentioned moulds, that have been prelubricated with an emulsifying agent and preheated to a temperature of 130° C., attaching the handles to the candy mass before the mass sets. It is left to cool for two hours to room temperature. Once the candy pieces are cold, they are removed from the moulds and wrapped or bagged.

Three hundred and fifty candy pieces with their handles are obtained. Their composition is:

| | |
|---|---|
| Sucrose | 2.5 Grams |
| Glucose | 1.4 Grams |
| H₂O | 0.5 Grams |
| Fentanyl Citrate | 400 μGrams |

1. A method for the production of fentanyl-based analgesics for their oral transmucosal administration in a candy-like dosage form, the method characterized by comprising the steps of:
a) Prepare a first solution by adding purified water to a vial containing Fentanyl citrate in an amount that allows the obtention of 370 μgrams to 430 μgrams solution per candy, shaking the vial until it is completely dissolved. Set aside;
b) Prepare a second solution by adding purified water to a vial containing a pharmacologically accepted dye, shaking the vial until it is completely dissolved. Set aside;
c) Mix polyalcohol sugar with purified water with a ratio polyalcohol/sugar between 17 and 19.5% by weight. Stir slowly over low heat until a liquid and smooth mass is obtained. Add sucrose to this liquid mass with a ratio liquid mass/sucrose between 65 and 67.5% by weight, and stir until the mixture is smooth.
d) Increase heat to high and bring to boil;
e) Maintain heat from an initial temperature of 128°-132° C. to a maximum temperature of 148°-152° C., with a gradual and controlled temperature increase gradient, stirring constantly to avoid the crystallization of sugar on the rim of the reactor or container, until water content, including the water contained in the polyalcohol sugar, evaporates and until the weight of the mass obtained in this step reaches between 88% and 90% by weight with respect to the initial total weight of the liquid mass obtained in step (c);
f) Leave the mass obtained in step (e) to cool to a temperature of about 130° C., stirring gently;
g) Add the solutions obtained in steps (a) and (b) and a flavouring agent, stirring firmly and maintaining the temperature reached in step (f);
h) Pour this hot mixture with a temperature of about 130° C. into a mould or matrix;
i) When the mould is cold and the candy has solidified, remove from the mould and immediately wrap or bag the candy pieces.

2. Method, as claimed in claim 1, characterized in that, after the hot mass is poured into a mould or matrix, at a temperature of about 130° C., according to step (h), a handle is attached to the soft mass in each mould and is left to cool.
